(19) United States
(12) Patent Application Publication
(10) Pub. No.: US 2019/0105331 A1
(43) Pub. Date: Apr. 11, 2019

(54) METHODS AND COMPOSITIONS FOR TREATING BROMHIDROSIS, HYPERHIDROSIS, HIDRADENITIS SUPPURATIVA AND RELATED CONDITIONS OF THE HAIR FOLLICLE

(71) Applicant: Adam M. Rotunda, Irvine, CA (US)

(72) Inventor: Adam M. Rotunda, Irvine, CA (US)

(21) Appl. No.: 16/154,804

(22) Filed: Oct. 9, 2018

Related U.S. Application Data
(60) Provisional application No. 62/569,790, filed on Oct. 9, 2017.

(51) Int. Cl. A61K 31/575; A61K 9/00; A61P 17/00

(52) U.S. Cl. CPC A61K 31/575; A61K 9/0019; A61K 9/06; A61P 17/00; A61K 9/0014

(57) ABSTRACT

Methods and compositions for alleviating treating bromhidrosis, hyperhidrosis, hidradenitis supportiva, and related conditions of the hair follicle are contemplated via the administration of at least one pharmacologically active bile acid to the area of skin containing the apocrine or eccrine glands causing such conditions. The detergent effect of the bile acid is operative to destabilize and permanently render these glands biologically inactive, substantially disrupting sweat production in the treated area, thus alleviating the treated condition. Topical and injectable formulations and methods of administrations are contemplated, along with various therapeutic regiments.

### CROSS-REFERENCE TO RELATED APPLICATIONS

This application relates to and claims the benefit of U.S. Provisional Application No. 62/569,790 filed Oct. 9, 2017 and entitled “METHODS AND COMPOSITIONS FOR TREATING BROMHIDROSIS AND HYPERHIDROSIS,” the entire disclosure of which is hereby wholly incorporated by reference.

### STATEMENT RE: FEDERALLY SPONSORED RESEARCH/DEVELOPMENT

Not Applicable

### BACKGROUND

### 1. Technical Field

The present disclosure relates generally to compositions and methods for treating bromhidrosis, hyperhidrosis, and hidradenitis supportiva. More particularly, the present disclosure relates to compositions and methods for treating bromhidrosis, hyperhidrosis, and hidradenitis supportiva which utilize bile acids, and in particular deoxycholate, as the active agents for disabling the function of eccrine and apocrine sweat glands causing such conditions.

### 2. Related Art

Bromhidrosis, hyperhidrosis, and hidradenitis supportiva are well-known medical conditions. Bromhidrosis is a condition characterized by excess sweating from apocrine sweat glands that are typically distributed in areas abundant in hair follicles, such as the arm pits and anogenital regions, as well as the breasts and buttocks. Apocrine glands have no thermoregulatory role but do secrete sweat that further includes fatty acids and proteins which make the sweat more viscous. Apocrine glands empty such sweat into hair follicles that ultimately reach the skin surface. While initially odorless, sweat produced by apocrine glands is subsequently subjected to bacterial decomposition that subsequently produces ammonia and short-chained fatty acids, which have characteristic strong and unpleasant odors, which can affect quality of life and negatively impact psychological, emotional and social well-being. Furthermore, these fatty acids produced by apocrine glands serve as both contributors of a biofilm that may result in or exacerbate hidradenitis suppurativa, clogging together bacteria and other substances that plug the hair follicle and contribute to inflammation.

Hyperhidrosis, in contrast, is a condition characterized by excessive sweating produced by eccrine glands. Per well-understood physiology, eccrine glands secrete sweat directly through a duct onto the surface of the skin that, through evaporative cooling, is operative to regulate body temperature. Hyperhidrosis is characterized by abnormally increased sweating in excess of that required for regulation of body temperature. In this regard, hyperhidrosis can either be generalized, or localized in specific parts of the body, such as the hands, feet, arm pits, groin and facial area where higher concentration of eccrine glands are situated. When such sweating occurs in such localized areas, the condition is referred to as primary hyperhidrosis or focal hyperhidrosis. Such condition, similar to bromhidrosis, is well-known to affect quality of life and negatively impact psychological, emotional and social well-being.

Hidradenitis suppurativa (HA) is a chronic inflammatory condition that usually begins in puberty and results when hair follicles and their associated glands in the areas of high apocrine and eccrine gland density (anogenital, axilla, breast, buttocks) become blocked and inflamed. The normal activity and structure of the hair follicle (including function or the apocrine and eccrine glands) is impaired and altered, which results in painful lumps, tunnels, and scarring due to the acute and chronic reaction of the body against the hair follicle and its related glandular structures.

Unfortunately, bromhidrosis, hyperhidrosis, and HA may be difficult to effectively treat and manage. With respect to bromhidrosis, treatment typically initially involves hygienic measures, such as adequate washing of the target areas of skin, from which the condition arises, as well as removal of sweaty clothing and the use of deodorants. In circumstances where basic hygienic measures are insufficient, regular shaving and deployment of electrolysis focusing on hair removal have also been utilized, as well as the use of topical antibiotics directed to limiting the growth of the contributory bacteria that decompose apocrine secretions. Drying agents have likewise been utilized to treat bromhidrosis. However, these treatments sometimes have a limited degree of effectiveness, and do not offer a definitive cure. Indeed, most methods for treating and managing bromhidrosis are deemed short lived and incomplete.

The management and treatment of hyperhidrosis likewise suffers from unsatisfactory options. In this regard, hyperhidrosis is typically treated with antiperspirant solutions or gels having an elevated concentration of aluminum chloride, the active ingredient utilized in most antiperspirants. In more severe cases anticholinergics are prescribed as they are operative to decrease sweating; however, such compounds are known to produce a host of adverse side effects, such as drowsiness, visual symptoms and dryness of the mouth, which makes the use of such agents suboptimal.

Still further, other known options for treatment and management of hyperhidrosis may include injections of botulism toxin type A, which is used to block neural control of sweat glands, or sweat gland removal or destruction via surgery. Such treatments, however, suffer from several drawbacks and can be painful and expensive. For example, ongoing injunctions of botulism toxin are required, as the effects of the toxin on a gland's function are temporary. Surgical removal or destruction of sweat glands, which may be accomplished via the use of lasers, energy devices, liposuction, or other methods, in many cases may be too intrusive or prohibitively expensive for many suffering from such conditions.

The management and treatment of HA suffers from similar defects. Conventional treatments include medications (steroids and other anti-inflammatory drugs topically, orally, and by injection), hormones, antibiotics, surgery, laser and liposuction (to destroy and remove the sweat glands), as well as biologic drugs that suppress the immune system. However, these options suffer from similar defects as known treatments for bromhidrosis and hyperhidrosis.

As such, there is a substantial need in the art for ways to effectively and efficiently treat the conditions of bromhidrosis, hyperhidrosis, HA, and other conditions of the hair follicle. There is likewise a need in the art for methods for treating bromhidrosis, hyperhidrosis, and HA, and related conditions that are permanent in nature, safe and easy to deploy, and will produce minimal side effects. There is further a substantial need in the art for compositions that are operative to effectively and permanently treat bromhidrosis, hyperhidrosis, HA, and related conditions that are not only exceptionally potent, but are also safe and capable of being readily and easily administered in order to treat such conditions.

### BRIEF SUMMARY

To solve these and other problems, compositions and methods for specifically address and alleviates the above-identified deficiencies in the art are contemplated. Such compositions contemplated for treating bromhidrosis, hyperhidrosis, and hidradenitis suppurativa may comprise a therapeutically effective amount of at least one bile acid or pharmaceutically acceptable salt thereof, the bile acid or pharmaceutically acceptable salt thereof being present in an amount operative to disrupt the sweat product of the apocrine glands or eccrine glands. Methods of application of such compositions are also contemplated, and may include application via topical application or injection in the area containing the apocrine glands or eccrine glands.

Specifically, a method for treating bromhidrosis is contemplated as comprising the step of applying a composition comprising an effective amount of at least one bile acid or pharmaceutically acceptable salt thereof upon an area of skin having apocrine glands lying there underneath, said at least one bile acid or pharmaceutically acceptable salt thereof being present in an amount operative to disrupt the sweat production of said apocrine glands. In an exemplary embodiment, the bile acid or pharmaceutically acceptable salt thereof comprises deoxycholate, which may be present in an amount in the composition ranging from 0.1% to 10% by weight, and which may be administered in an amount ranging from 20 to 2000 mg/square decimeter of skin, or more preferable, in an amount ranging from 100 to 500 mg/square decimeter of skin.

A pharmaceutical composition for topical application is also contemplated, the pharmaceutical composition comprising a therapeutically effective amount of at least one bile acid salt, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, wherein the bile acid salt is present in an amount ranging from 0.1% to 10% by weight of said composition, and wherein said composition is formulated as a cream, lotion, ointment, gel, foam, wax or spray. According to a preferred embodiment of such a pharmaceutical composition, the bile acid salt may comprise sodium deoxycholate, and/or may be incorporated as part of a transdermal delivery mechanism.

A method for treating hyperhidrosis is also contemplated, the method comprising the step of injecting a composition containing at least one bile acid salt within an area of skin containing eccrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.

A further method for treating hyperhidrosis is also contemplated, the method comprising topically applying a composition containing a therapeutically effective amount of at least one bile acid salt upon an area of skin having eccrine glands, said at least one bile acid salt being present in an amount operative to disrupt the sweat production of said eccrine glands. According to preferred embodiments of this method, the at least one bile acid salt may comprise sodium deoxycholate, which may be present in said composition in amounts ranging from 0.1% to 10% by weight, and which may also be topically applied upon said area of skin in an amount ranging from 20 to 2000 mg/square decimeter of skin, or more preferably, in an amount ranging from 100 to 500 mg/square decimeter of skin.

A further method for treating bromhidrosis is also contemplated, the method comprising injecting a composition containing at least one bile acid salt within an area of skin containing apocrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.

A method of treating hidradenitis suppurativa is also contemplated, the method comprising the step of injecting a composition containing at least one bile acid salt within an area of skin containing eccrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.

A further method for treating HA is also contemplated, the method comprising topically applying a composition containing a therapeutically effective amount of at least one bile acid salt upon an area of skin having eccrine glands, said at least one bile acid salt being present in an amount operative to disrupt the sweat production of said eccrine glands. According to preferred embodiments of this method, the at least one bile acid salt may comprise sodium deoxycholate, which may be present in said composition in amounts ranging from 0.1% to 10% by weight, and which may also be topically applied upon said area of skin in an amount ranging from 20 to 2000 mg/square decimeter of skin, or more preferably, in an amount ranging from 100 to 500 mg/square decimeter of skin

### DETAILED DESCRIPTION

According to various aspects of the present disclosure, methods and compositions for addressing and alleviating bromhidrosis, hyperhidrosis, and hidradenitis supportiva (HA) are contemplated via the administration of at least one pharmacologically active bile acid to the area of skin containing the apocrine or eccrine glands causing such conditions, such that the detergent effect of the bile acid is operative to structurally destabilize and permanently render such glands biologically inactive. This may result in the disruption and possibly the permanent cessation of the production of sweat in the treated area, and consequently treatment of the underlying conditions.

Throughout this disclosure, reference is made to bile acids useful for the practice of the presently disclosed methods and compositions. Such bile acids the same may be any one or any combination of bile acids or bile salts that are operatively converted into either primary bile acids, similar to those synthesized by the liver, or secondary bile acids produced from bacterial actions in the colon, all of which are readily understood and may be readily formulated by those skilled in the art. For example, a bile salt may at least one of a deoxycholic, a cholic, a chenodeoxycholic, a 7-alpha-dehydroxylate, a chenodeoxycholic, a lithocholic, a ursodeoxycholic, a dihydroxy, or a trihydroxy-bile salt. Such bile acids or bile salts may also be in the taurine or glycine conjugate forms. For ease of reference throughout this disclosure, all of these shall be referred to under the term “bile acid” which is to be understood as inclusive of all of these potential variations and configurations.

According to a preferred exemplary embodiment, the bile acid used in the contemplated methods and compositions may be deoxycholic acid and/or its corresponding sodium salt, sodium deoxycholate. Deoxycholate has the chemical structure shown below:

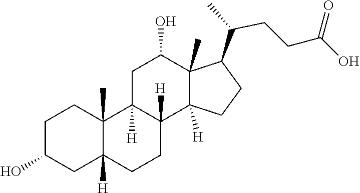

Deoxycholate, along with other bile acids, are operate to lyse and emulsify lipids, and have proven to be effective in dissolving fatty tissues whereby the detergent-like effect of such compositions disrupts and disperses lipid materials, including the lipids that constitute cell membranes. As a consequence, the structural integrity and biological function of the biological components are neutralized. It may therefore be seen that the application of a bile acid like deoxycholate may effectuate the disablement of both apocrine and eccrine glands in a manner that may be selectively targeted and achieved based upon the application of the active component to the area of the body containing the target glands of interest.

To effectuate this end, it is contemplated that the presently disclosed compositions may be applied topically or by subcutaneous injection at the specific location where it is desired to attain the local effect of disruption of sweat production by apocrine and eccrine glands, with the goal of effectuating permanent disruption of effectively all sweat production by such glands in the targeted area. To produce the desired disablement of apocrine glands in order to treat bromhidrosis, it is contemplated that the bile acid, including deoxycholate by itself or in combination with other bile acids, may be applied in amounts ranging from 20 to 2000 mg/square decimeter of skin, and in a more refined embodiment, applied in amounts from 100 to 500 mg/square decimeter of skin, and in a most highly preferred embodiment, it is administered in an amount ranging from 150 to 250 mg/square decimeter of skin to be treated.

According to one preferred formulation of a treatment for bromhidrosis, it is contemplated that deoxycholate may be applied via a pharmaceutical composition wherein the deoxycholate component will be present in the composition in an amount ranging from 0.1% to 10% by weight, and in a more highly preferred embodiment, from 0.5% to 1.5% by weight, whereby the remaining ingredients will comprise inactive materials. The composition will also preferably be formed as a cream, lotion, ointment, gel, foam, wax, spray or other formulation for ease of topical application.

These compositions for the treatment of bromhidrosis may be applied, according to an exemplary regimen, once every several weeks. However, it may also be seen that other regimens are possible, including applications ranging from once a week to once every 4-8 weeks, for multiple consecutive months (ranging from 1-6 months). However, it may be seen that in certain embodiments, other regimens of treatment may be contemplated, including one-time treatments, or treatments of an ongoing, indeterminate nature.

With respect to the treatment of hyperhidrosis, caused primarily by sweat production form eccrine glands, it is contemplated that the compositions of the present disclosure may be applied such that the bile acid, and in particular the deoxycholate, must be applied in amounts ranging from 20 to 2000 mg/square decimeter of skin, and in a more refined embodiment, applied in amounts from 100 to 500 mg/square decimeter of skin, and in a most highly preferred embodiment, it is administered in an amount ranging from 150 to 250 mg/square decimeter of skin to be treated.

According to one preferred formulation of a treatment for hyperhidrosis, it is contemplated that deoxycholate may be applied via a pharmaceutical composition wherein the deoxycholate component will be present in the composition in an amount ranging from 0.1% to 10% by weight, and in a more highly preferred embodiment, from 0.5% to 1.5% by weight, whereby the remaining ingredients will comprise inactive materials. The composition will also preferably be formed as a cream, lotion, ointment, gel, foam, wax, spray or other formulation for ease of topical application.

Such compositions for the treatment of hyperhidrosis may be applied according to an exemplary regimen of once every several weeks, ranging from once a week to once every 4-8 weeks, for multiple consecutive months (ranging from 1-6 months). However, it may be seen that in certain embodiments, other regimens of treatment may be contemplated, including one-time treatments, or treatments of an ongoing, indeterminate nature.

Alternatively, to the extent desired, the compositions herein contemplated may be deployed through a conventional transdermal drug delivery system, such as a transdermal patch using transdermal delivery mechanisms well-known in the art. In this regard, the compositions of the present invention, as well as the manner by which the same are applied, are specifically formulated for localized application on discrete areas of the body where localized apocrine and/or eccrine glands are sought to be treated and deactivated, and are specifically formulated and intended to be applied in a manner that does not involve any systemic distribution of the active bile acid agent or agents.

With respect to the treatment of HA caused primarily by sweat production form eccrine glands, it is contemplated that the compositions of the present disclosure may be applied such that the bile acid, and in particular the deoxycholate, must be applied in amounts ranging from 20 to 2000 mg/square decimeter of skin, and in a more refined embodiment, applied in amounts from 100 to 500 mg/square decimeter of skin, and in a most highly preferred embodiment, it is administered in an amount ranging from 150 to 250 mg/square decimeter of skin to be treated.

According to one preferred formulation of a treatment for HA, it is contemplated that deoxycholate may be applied via a pharmaceutical composition wherein the deoxycholate component will be present in the composition in an amount ranging from 0.1% to 10% by weight, and in a more highly preferred embodiment, from 0.5% to 1.5% by weight, whereby the remaining ingredients will comprise inactive materials. The composition will also preferably be formed as a cream, lotion, ointment, gel, foam, wax, spray or other formulation for ease of topical application.

Such compositions for the treatment of HA may be applied according to an exemplary regimen of once every several weeks, ranging from once a week to once every 4-12 weeks, for multiple consecutive months (ranging from 1-6 months). However, it may be seen that in certain embodiments, other regimens of treatment may be contemplated, including one-time treatments, or treatments of an ongoing, indeterminate nature.

In order to formulate a specific topical pharmaceutical composition operative to effectively administer the destabilizing amounts of bile acid detergent sufficient to disrupt the sweat production functionality of apocrine or eccrine glands, the compositions herein contemplated may be formulated per any of a variety of known techniques that enable the bile acid to be topically deployed. Accordingly, additional inactive ingredients that can be mixed and formulated as lotions, creams, ointments and the like may be utilized in the formulation of such pharmaceutical compositions.

In an alternative embodiment, the compositions herein contemplated for treatment of bromhidrosis and/or hyperhidrosis may be formulated as an injectable composition wherein the bile acid may be injected in a concentration ranging from to 20 to 2000 mg/square decimeter of surface area of skin to be treated. In a more refined embodiment, the bile acid salt may be injected in an amount from 100 to 500 mg/square decimeter of skin treated. In this regard, it is believed that these discussed concentrations of bile acid may apply to any of those bile acids discussed herein, and in particular with respect to deoxycholate.

With respect to the administration of such injectable compositions, it may be desirable to inject subcutaneously directly into the area of skin to be treated. The injection may be administered as a single injection, or may be administered as multiple injections across the area of skin where treatment is desired. To that end, it is contemplated that the injectable embodiments of the presently disclosed compositions may be applied according to an exemplary regimen of once every several weeks, ranging from once a week to once every 4-8 weeks, for multiple consecutive months (ranging from 1-6 months). However, it may be seen that in certain embodiments, other regimens of treatment may be contemplated, including one-time treatments, or treatments of an ongoing, indeterminate nature.

Along those lines, it is contemplated that the above exemplary regimens of an injectable composition may be well-suited for the treatment of bromhidrosis, such that the apocrine glands of a given area of skin may be substantially or completely disabled over the course of a therapeutic regimen, and it is further contemplated that the above exemplary regimens of an injectable composition may be well-suited for the treatment of hyperhidrosis, such that the eccrine glands of a given area of skin are substantially or completely disabled over the such course of a therapeutic regimen. It is further contemplated that the above exemplary regimens of an injectable composition may be well-suited for the treatment of HA, such that the eccrine glands of a given area of skin are substantially or completely disabled over the course of a therapeutic regimen.

As discussed above, it is understood that the bile acid component is, in fact, the sole active ingredient necessary to effectuate treatment of bromhidrosis and hyperhidrosis via the functional disablement of apocrine and eccrine glands. No other active ingredients are necessary. It should be understood, however, that the injectable compositions presently contemplated and disclosed, as well as the topical compositions herein contemplated and disclosed, may be mixed with other suitable carriers and/or excipients and formulated per conventional pharmaceutical manufacturing practices. To this end, it is contemplated that other pharmaceutical components may be included as a component of the herein disclosed injectable and/or topical formulations, with such other pharmaceutical compositions operative to impart one or more further desired physiological and/or therapeutic effects. Such other pharmaceutical compounds may include, without limitation, anti-inflammatories, antibiotics, anesthetics, dispersing agents (such as hyaluronidase) and the like.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the exemplary embodiments.

What is claimed is:

1. A method for treating bromhidrosis comprising applying a composition containing an effective amount of at least one bile acid or pharmaceutically acceptable salt thereof upon an area of skin having apocrine glands lying there underneath, said at least one bile acid or pharmaceutically acceptable salt thereof being present in an amount operative to disrupt the sweat production of said apocrine glands.

2. The method of claim 1 wherein said at least one bile acid or pharmaceutically acceptable salt thereof comprises deoxycholate.

3. The method of claim 2 wherein said deoxycholate is present in said composition in amounts ranging from 0.1% to 10% by weight.

4. The method of claim 2 wherein said deoxycholate is administered in an amount ranging from 20 to 2000 mg/square decimeter of skin.

5. The method of claim 4 wherein said deoxycholate is administered in an amount ranging from 100 to 500 mg/square decimeter of skin.

6. A pharmaceutical composition for topical application comprising:
a therapeutically effective amount of at least one bile acid salt, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid;
wherein the bile acid salt is present in an amount ranging from 0.1% to 10% by weight of said composition; and
wherein said composition is formulated as a cream, lotion, ointment, gel, foam, wax or spray.

7. The composition of claim 6 wherein said bile acid salt comprises sodium deoxycholate.

8. The composition of claim 7 wherein said composition is incorporated as part of a transdermal drug delivery system.

9. A method for treating hyperhidrosis comprising injecting a composition containing at least one bile acid salt within an area of skin containing eccrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.

10. A method for treating hyperhidrosis comprising topically applying a composition containing a therapeutically effective amount of at least one bile acid salt upon an area of skin having eccrine glands, said at least one bile acid salt being present in an amount operative to disrupt the sweat production of said eccrine glands.

11. The method of claim 10 wherein at least one bile acid salt comprises sodium deoxycholate.

12. The method of claim 11 wherein said sodium deoxycholate is present in said composition in amounts ranging from 0.1% to 10% by weight.

13. The method of claim 11 wherein said sodium deoxycholate is topically applied upon said area of skin in an amount ranging from 20 to 2000 mg/square decimeter of skin.

14. The method of claim 13 wherein said sodium deoxycholate is administered in an amount upon said skin ranging from 100 to 500 mg/square decimeter of skin.

15. A method for treating bromhidrosis comprising injecting a composition containing at least one bile acid salt within an area of skin containing apocrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.

16. A method for treating hidradenitis supportiva comprising topically applying a composition containing a therapeutically effective amount of at least one bile acid salt upon an area of skin having apocrine or eccrine glands, said at least one bile acid salt being present in an amount operative to disrupt the sweat production of said apocrine or eccrine glands.

17. The method of claim 16 wherein at least one bile acid salt comprises sodium deoxycholate.

18. The method of claim 17 wherein said sodium deoxycholate is present in said composition in amounts ranging from 0.1% to 10% by weight.

19. The method of claim 17 wherein said sodium deoxycholate is topically applied upon said area of skin in an amount ranging from 20 to 2000 mg/square decimeter of skin.

20. The method of claim 19 wherein said sodium deoxycholate is administered in an amount upon said skin ranging from 100 to 500 mg/square decimeter of skin.

21. A method for treating hidradenitis supportiva comprising injecting a composition containing at least one bile acid salt within an area of skin containing apocrine or eccrine glands, the at least one bile acid salt being a pharmaceutically acceptable salt of one or more bile acid chosen from the group of: deoxycholic acid, cholic acid, chenodeoxycholic acid, 7-alpha-dehydroxylate, chenodeoxycholic acid, lithocholic acid, ursodeoxycholic acid, a dihydroxy bile acid, or a trihydroxy bile acid, the at least one bile acid salt being injected in a concentration ranging from 20 to 2000 mg/square decimeter of surface area of skin to be treated.
